(19) United States
(12) Patent Application Publication
(10) Pub. No.: US 2005/0288630 A1
(43) Pub. Date: Dec. 29, 2005

(54) Cuff resistant foley catheter

(76) Inventor: Anthony J. Conway, Chatfield, MN (US)

Correspondence Address: Merchant & Gould P.C., P.O. Box 2903, Minneapolis, MN, 55402-0903, US

(21) Appl. No.: 11/170,194

(22) Filed: Jun. 29, 2005

Related U.S. Application Data
(63) Continuation-in-part of application No. 11/039,074, filed on Jan. 20, 2005.
(60) Provisional application No. 60/539,054, filed on Jan. 22, 2004.

(51) Int. Cl. A61M 29/00

(52) U.S. Cl. 604/96.01; 604/544

(57) ABSTRACT

The invention relates to a Foley-type catheter constructed to prevent a retention balloon of the Foley catheter from cuffing. The retention balloon has a length of less than or equal to 0.40 inches. A kit, including the cuff-resistant Foley catheter and a syringe containing about 6 cc of water, can be provided.

### CROSS REFERENCE TO RELATED APPLICATIONS

This application is a continuation-in-part of application Ser. No. 11/039,074, filed Jan. 20, 2005, which application claims benefit of Provisional Application No. 60/539,054, filed Jan. 22, 2004; which applications are incorporated herein by reference.

### FIELD OF THE INVENTION

The invention relates to a Foley-type catheter having a retention balloon. More particularly, the invention relates to a catheter with a retention balloon made of silicone rubber.

### BACKGROUND OF THE INVENTION

Foley-type catheters are tube-like devices that are used to drain urine from a patient's bladder. Foley catheters are inserted through the urethra and typically held in place with an inflatable balloon. The balloon is in a deflated position when the catheter is first inserted. Then, once the catheter is in the proper position, the balloon is inflated with a fluid. The inflated balloon is larger in diameter than the diameter of the urethra and thereby physically inhibits movement of the catheter. Foley catheters are also known as “indwelling” catheters because they are designed to be left in place for a period of time.

Latex rubber is most often used in the manufacture of Foley catheters. However, latex rubber can be problematic as many patients have latex allergies. To provide an alternative for patients with allergies, silicone rubber has since been used to make Foley catheters. Silicone rubber does not, however, have the same elastic properties as latex rubber. As a result, balloons of Foley catheters made with silicone rubber can exhibit “cuffing.”

Cuffing refers to the situation in which the balloon tends to fold over on itself or shift toward the bladder end of the catheter. Because the balloon is attached at its end to the shaft of the catheter, a cuff forms when the outer expanded portion of the balloon pushes over the inner attached end of the balloon. This cuff can remain when the balloon is deflated before withdrawal of the catheter from the patient. The cuff results in the deflated balloon having a larger diameter than it did when it was first inserted. The increased diameter can result in discomfort and injury to patients. Accordingly, a need exists for a silicone rubber Foley catheter that resists cuffing.

### SUMMARY OF THE INVENTION

One aspect of the present disclosure relates to catheter including a catheter shaft and an inflatable balloon. The catheter shaft defines a first lumen and a second lumen, the first lumen being in fluid communication with an opening located at a distal end of the catheter shaft. The inflatable balloon is positioned in fluid communication with the second lumen of the catheter shaft. The inflatable balloon has a length defined between a first end of the balloon attached to the catheter shaft and a second end of the balloon attached to the catheter shaft. The length of the balloon is about 0.40 inches or less.

Another aspect of the present disclosure relates to a kit including a catheter and a pre-filled syringe. The catheter includes a catheter shaft defining a fluid lumen and a capillary lumen, a balloon in fluid communication with the capillary lumen, and an end piece having first and second ports in fluid communication with the fluid and capillary lumen. The syringe is pre-filled with less than about 7 cc of fluid, and is constructed to correspond to the configuration of the second port to permit fluid communication between the syringe and the balloon.

Still another aspect of the present disclosure relates to a method of manufacturing a catheter having a catheter shaft and an inflatable retention balloon attached to the shaft at first and second ends of the balloon. The balloon has a length between the first and second ends of about 0.40 inches or less.

A variety of examples of desirable product features or methods are set forth in part in the description that follows, and in part will be apparent from the description, or may be learned by practicing various aspects of the disclosure. The aspects of the disclosure may relate to individual features as well as combinations of features. It is to be understood that both the foregoing general description and the following detailed description are explanatory only, and are not restrictive of the claimed invention.

### BRIEF DESCRIPTION OF THE FIGURES

FIG. 1A is a schematic view of a catheter is an original deflated configuration;

FIG. 1B is a schematic view of a catheter in an inflated position wherein the balloon is cuffing;

FIG. 1C is a schematic view of a catheter in a deflated position wherein the balloon has retained a cuff;

FIG. 2 is a partial cross-sectional view of a retention balloon of a Foley catheter made in accordance with the principles of the present disclosure;

FIG. 3 is a cross-sectional view of an embodiment of a retention balloon having ribs;

FIG. 4 is a partial cross-sectional view of the retention balloon of the Foley catheter of FIG. 2, shown with the retention balloon of the catheter expanded;

FIG. 5 is a partial cross-sectional view of the Foley catheter of FIG. 2, shown with the retention balloon and an end piece; and

FIG. 6 is a schematic illustration of a kit including the Foley catheter of FIG. 5 and a syringe.

### DETAILED DESCRIPTION OF THE INVENTION

Cuffing

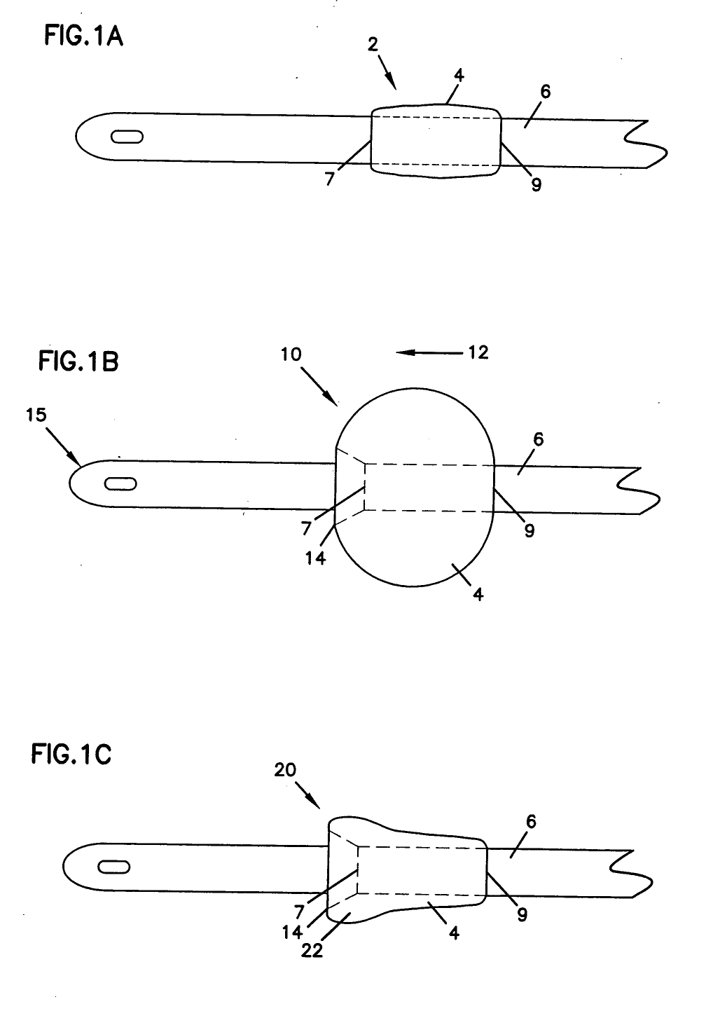

As described above, balloon catheters made with silicone rubber can exhibit problematic cuffing. FIG. 1A shows a schematic view of a catheter in a deflated configuration 2. The catheter includes a balloon 4 and a catheter shaft 6. In the deflated configuration 2, the balloon 4 does not overlap either its distal end 7 or its proximal end 9. Further, in the configuration shown in FIG. 1A, the balloon 4 adds only a small increment to the diameter of the catheter shaft 6 because of how the uninflated balloon 4 lies flat over the catheter shaft 6.

However, as described above, balloon catheters made with silicone rubber may exhibit problems with cuffing. FIG. 1B is a schematic view of a catheter in an inflated configuration 10 wherein the balloon 4 is cuffing. Cuffing refers to the situation in which the balloon 4 tends to be shifted toward the bladder end 15 of the catheter (in the direction of arrow 12) forming a cuff 14, as the balloon 4 itself is pressed against the bladder wall when holding the catheter in place. Since the balloon 4 is attached at its distal end 7 to the catheter shaft 6, the balloon forms a cuff 14 as the outer expanded portion of the balloon 4 is pushed over the inner attached distal end 7 of the balloon 4.

The cuff 14 that forms tends to remain when the balloon 4 is deflated. FIG. 1C is a schematic view of a catheter in a deflated configuration 20 after having been inflated wherein the balloon formed a cuff 14. The cuff 14 results in the deflated balloon 4 having a larger diameter in an area 22 of the balloon 4 over the cuff 14 than when first inserted. A balloon that has cuffed may be 12 French sizes larger at the cuff, for example, than the actual catheter shaft 6. The increased diameter can result in discomfort and injury to patients.

Cuff Resistant Catheters

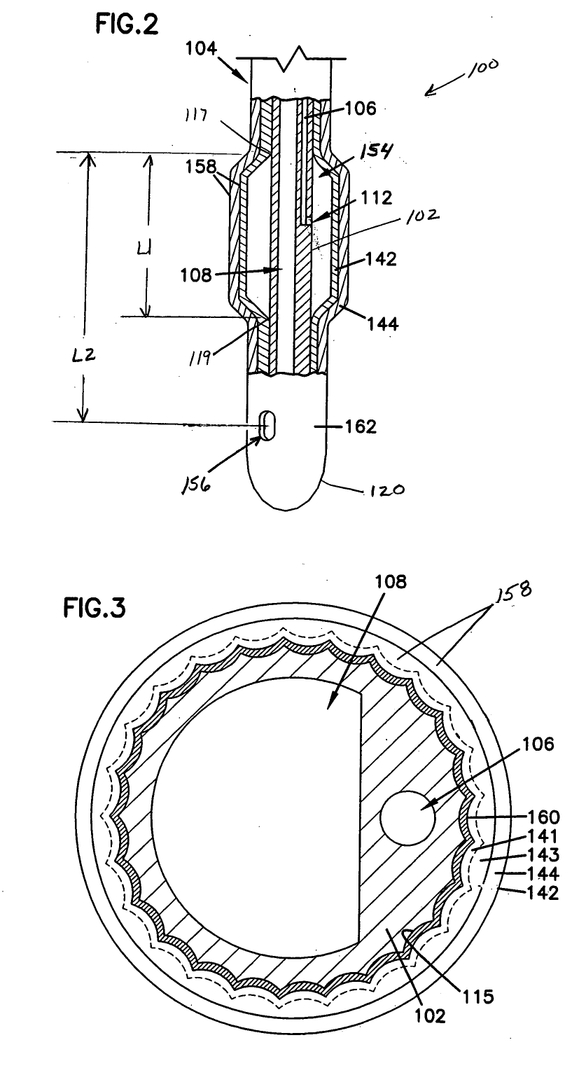

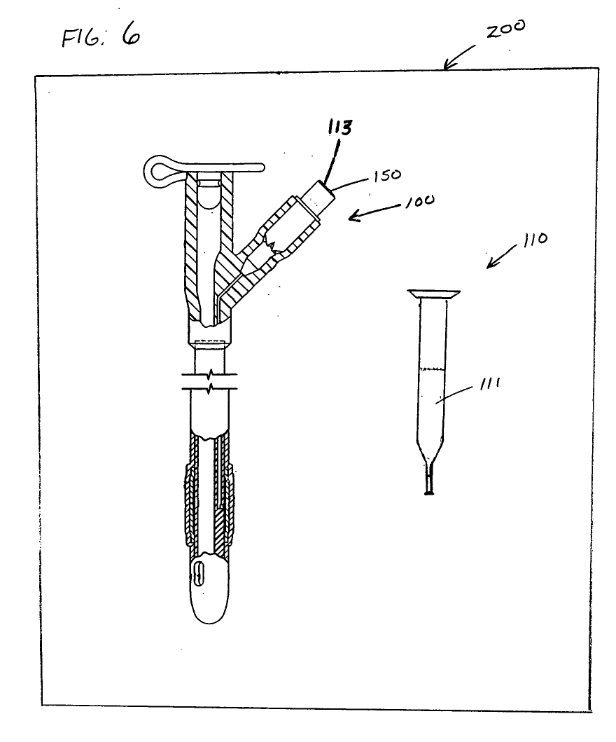

Referring now to FIGS. 2-5, the present inventors have created embodiments of Foley catheters (e.g., 100) that resist cuffing. In an embodiment of the invention, the Foley catheter 100 includes a catheter shaft 104 (FIG. 2) and an end piece 146 (FIG. 5). The catheter shaft 104 includes a retention balloon 158.

Referring to FIG. 2, a partial cross-sectional view of a portion of the shaft 104 and the retention balloon 158 of the Foley catheter 100 are illustrated. The catheter shaft 104 of the Foley catheter 100 is constructed from a double lumen tube 102. The double lumen tube 102 defines a fluid conduit lumen 108 and a capillary lumen 106. The retention balloon 158 of the catheter shaft 104 is a multi-layer retention balloon. In particular, the retention balloon 158 is constructed of two layers formed on the double lumen tube 102: a balloon layer 142 and a sheath layer 144. In one embodiment, each of the balloon layer 142 and the sheath layer 144 is made of silicone rubber, such as Dow Corning C6-515 or other suitable polymeric bonding compositions.

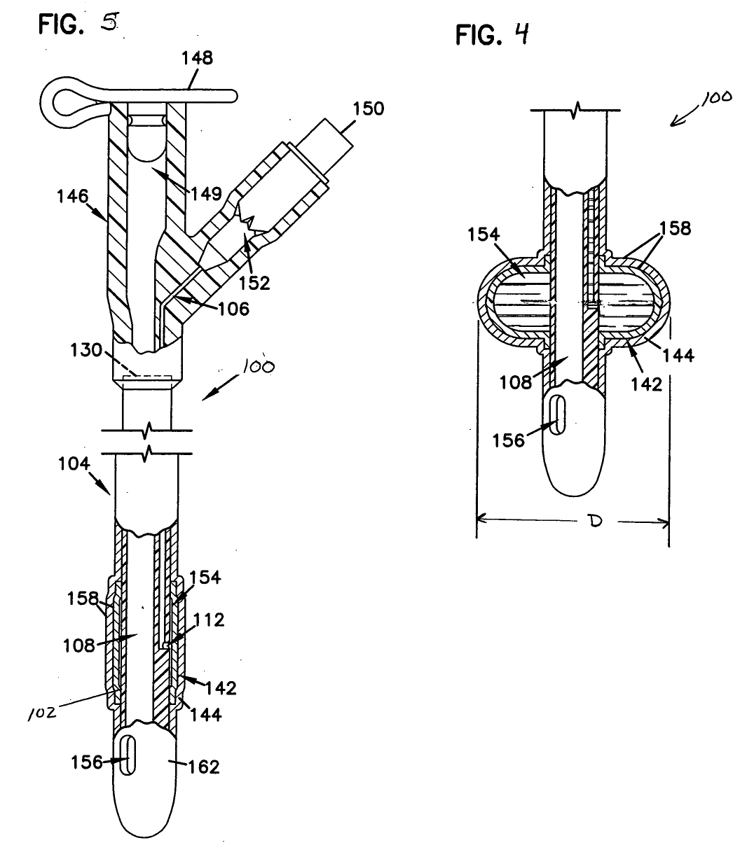

The multi-layer retention balloon 158 of the Foley catheter 100, including the balloon layer 142 and the sheath layer 144, is not bonded to the double lumen tube 102. Rather, a balloon cavity 154 is disposed under the balloon layer 142. The balloon cavity 154 is in fluid communication with the capillary lumen 106 via a capillary lumen access opening 112 formed in the double lumen tube 102. When a fluid is pumped or injected into the capillary access lumen 106, the retention balloon 158 and the balloon cavity 154 expand, as shown in FIG. 4.

In an embodiment, the balloon layer 142 of the Foley catheter 100 is an integral part of the catheter shaft 104. That is, the balloon layer 142 is an integral part of the catheter shaft 104 by the dipping and stripping methods described in U.S. patent application Ser. No. ______ (having Attorney Docket No. 8740.109US01), which application is incorporated herein by reference. In contrast, a balloon layer formed from cured material that is applied in cured form to a shaft (e.g., as a preformed sleeve or as a tape wound around the shaft) is not an integral part of a catheter shaft.

As shown in FIG. 5, the balloon layer 142 is provided only along a portion of the catheter shaft 104. The sheath layer 144 of the catheter shaft 104 is formed over the entire length of the shaft. In this manner, the difference between the diameter of the retention balloon 158 and the diameter of the catheter shaft 104 can be controlled simply by adjusting the thickness of the balloon layer 142. While not intending to be bound by theory, it is believed that the added thickness at the retention balloon 158 of the Foley catheter 100 resists balloon cuffing. Adding thickness to the retention balloon 158 also results in a stronger balloon that is less likely to burst.

In certain embodiments, the effective diameter of the shaft 104 and the diameter of the retention balloon 158 are increased commensurately so that the differential between the shaft and retention balloon are kept to an advantageous small amount, for example, about 4 French sizes (e.g., about 0.052 inch) or less than or equal to 4 French sizes (0.052 inch).

Referring back to FIG. 2, the retention balloon 158 of the Foley catheter 100 has a length L1 that extends from a first end 117 of the retention balloon 158 to a second end 119 of the retention balloon 158. Each of the first and second ends 117, 119 of the retention balloon 158 are defined at a region where the balloon layer 142 attaches to or is integral with the double lumen tube 102. In conventional arrangements, Foley balloons are typically 0.60 inches in length. In the present invention, the length L1 of the retention balloon 158 is preferably less than 0.45 inches; more preferably less than or equal to 0.40 inches.

In providing a retention balloon 158 having a shortened length L1, the occurrence of cuffing is reduced. Generally, balloons having the longer length relative to a particular balloon diameter have more length that can stretch and fold over to form a cuff. The shortened length L1 of the present invention reduces the likelihood of cuffing by lessening the amount of length that can stretch, thereby lessening the likelihood that the stretched length will fold over.

Because there is less balloon length or material that can stretch, the occurrence of regions experiencing inelastic deformation due to a weakened region in the balloon layer 142 is also reduced. The shortened length L1 of the retention balloon 158 thereby further provides more complete balloon return.

Referring now to FIG. 4, although the length L1 (FIG. 2) of the presently disclosed catheter 100 is shortened, the inflated diameter D of the retention balloon 158 of the Foley catheter 100 preferably remains the same as compared to conventional balloons. In particular, the retention balloon 158 of the Foley catheter 100 is constructed to expand or inflate to about 0.80 inches to 1.1 inches in diameter D so that the Foley catheter 100 remains in place during use.

As shown in FIG. 4, when inflated, the shortened length L1 (FIG. 2) of the Foley catheter 100 provides a retention balloon 158 that is donut-shaped or ring-shaped. The ring-shaped configuration is shorter in length than conventional arrangements to prevent cuffing, but sufficient in diameter to hold the catheter 100 in place. The retention properties of the ring-shaped configuration are undiminished in comparison to conventional Foley catheters, but the discomfort and possibility of injury caused by cuffing during removal of the catheter are significantly reduced. In the illustrated Foley catheter 100 embodiment, the length L1 to diameter D ratio is no more than about 1.0 to 2.0; more preferably about 1.0 to 3.0.

Referring again to FIG. 2, the Foley catheter 100 of the present disclosure includes a drainage eye or fluid conduit access opening 156 located through an exterior surface 162 of the catheter shaft 104. The drainage eye 156 is in fluid communication with the fluid conduit lumen 108.

The drainage eye 156 of the catheter shaft 104 is located a distance L2 from the retention balloon 158 of the catheter 100. The distance L2 is defined as the distance between the first end 117 of the retention balloon 158 and a general centerline of the drainage eye 156. In use, because of the shortened length L1 of the retention balloon 158, the drainage eye is located closer to the bladder neck of the patient's bladder in comparison to conventional arrangements. Providing an arrangement wherein the drainage eye 156 is closer to the patient's bladder neck results in more complete draining of the patient's bladder.

Also, because the drainage eye 156 is located a distance closer to the patient's bladder neck than conventional arrangement, a tip 120 of the catheter is not required to be inserted as far into the patient's bladder as compared to conventional arrangement. The required insertion depth for the presently disclosed Foley catheter 100 is reduced, and thereby the catheter 100 is less likely to contact and irritate the back of the patient's bladder wall.

In the illustrated embodiment, the distance L2 of the first end of the balloon 117 and the general centerline of the drainage eye 156 is preferably less than about 1.0 inches; more preferably less than or equal to about 0.80 inches.

Referring now to FIG. 3, in one embodiment, the balloon layer 142 of the retention balloon 158 is formed with ribs 160. It is intended that various embodiments of the present invention can include only one, or a combination of the features herein described, including: the multi-layered retention balloon 158, the shortened balloon length L1, and the ribs 160.

The ribs 160 are preferably made of a compound different from that of the sheath layer 144. In the illustrated embodiment, the balloon layer 142 has a first region 141 and a second region 143. The first region 141 includes the ribs 160. The ribs 160 are made of a less pliable silicone rubber than the silicone rubber of the second region 143 of the balloon layer 142, and than the silicone rubber of the sheath layer 144. That is, the compound of the ribs 160 preferably stretches less easily than the compound of the second region 143 of the balloon layer 142 and the compound of the sheath layer 144. The ribs 160 resist excessive balloon stretching and enhance the structural memory or return of the retention balloon so that the retention balloon 158 is less likely to cuff. While not intending to be bound by theory, it is further believed that positioning the ribs 160 to extend in a direction parallel to the catheter shaft 104 limits stretching of the retention balloon 158 in that direction, further aiding in the resistance of cuffing.

In the illustrated embodiment of FIG. 3, the ribs 160 fit within corresponding grooves 115 (e.g., undulations or channels) formed in the double lumen tube 102 of the catheter 100. When the retention balloon 158 is deflated, the ribs 160 rest within the grooves 115 of the double lumen tube 102 so that the diameter of the retention balloon 158 is not affected by the existence of the ribs.

Referring now to FIG. 5, the end piece 146 of the Foley catheter 100 is secured to a proximal end 130 of the catheter shaft 104. The end piece 146 defines a first access opening 149 and a second access opening 152. The first access opening 149 of the end piece 146 is in fluid communication with the fluid conduit lumen 108 of the catheter shaft 104. The second access opening 152 is in fluid communication with the capillary lumen 106. In the illustrated embodiment, a cap 148 is provided for closure of the first access opening 149. A luer valve 150 is also provided. The luer valve 150 is adapted for engagement into and closure of the second access opening 152.

In use, fluid (such as water) is injected into the capillary lumen 106 through the luer valve 150. The fluid flows through the capillary lumen access opening 112 into the balloon cavity 154 to expand the retention balloon 158. In conventional arrangements, the balloons having the standard length of 0.60 inches are typically inflated with 10 cc of water. Most conventional Foley catheters are sold in kits having syringes pre-filled with 10 cc of water.

As can be understood, reducing the length L1 of the disclosed retention balloon 158, while maintaining the size of the expanded balloon diameter D, decreases the volume of the balloon cavity 154. Accordingly, a lesser amount of fluid is required to expand the retention balloon 158 of the present Foley catheter 100. Referring now to FIG. 6, the present Foley catheter 100 can be provided in a kit 200. The kit includes the Foley catheter 100 having the shortened length L1 (FIG. 2) and a prefilled syringe 110 (schematically represented). The syringe 110 is pre-filled with about 6 cc of water 111, preferably less than about 7 cc of water 111. To remind the clinician that only 6 cc of water is needed to inflate the retention balloon 158, the Foley catheter 100 can include a sticker, markings, or other indicia 113. In the illustrated embodiment, the indicium 113 is located on the luer valve 150 of the Foley catheter.

Because of the smaller configuration and volume of the retention balloon 158 of the present Foley catheter 100, the weight of the retention balloon 158 is reduced, especially when filled with water. The disclosed Foley catheter 100 not only lessens the likelihood of discomfort or injury due to cuffing, the reduced balloon weight of the present Foley catheter 100 also lessens the likelihood of general trauma and irritation to the patient during indwelling periods.

The above specification provides a complete description of the. Since many embodiments of the invention can be made without departing from the spirit and scope of the invention, certain aspects of the invention reside in the claims hereinafter appended.

1. A catheter comprising:
a) a catheter shaft defining a first lumen and a second lumen, the first lumen being in fluid communication with an opening located at a distal end of the catheter shaft; and
b) an inflatable silicone balloon arranged in fluid communication with the second lumen of the catheter shaft, the inflatable balloon having a first end attached to the catheter shaft and a second end attached to the catheter shaft, the inflatable balloon having a length defined between the first end and the second end, the length of the balloon being about 0.40 inches or less.

2. The catheter of claim 1, wherein the first lumen is a fluid lumen sized to convey fluid from a patient's bladder through the catheter shaft, and wherein the second lumen is a capillary lumen sized to transport fluid to and from the inflatable balloon to inflate and deflate the balloon.

3. The catheter of claim 1, wherein the inflatable balloon has a diameter of about 0.80 inches to 1.1 inches when inflated.

4. The catheter of claim 1, wherein the inflatable balloon has a ring-shaped configuration when inflated.

5. The catheter of claim 4, wherein the balloon has a diameter, the balloon having a diameter to length ratio of about 1.0 to 3.0 when inflated.

6. The catheter of claim 1, wherein the inflatable balloon defines a balloon cavity, the balloon cavity being configured to receive less than about 7 cc of fluid.

7. The catheter of claim 6, wherein the balloon cavity is configured to receive about 6 cc of fluid.

8. The catheter of claim 1, wherein a centerline of the opening of the catheter shaft is located no more than 1.0 inches from the first end of the inflatable balloon.

9. The catheter of claim 8, wherein a centerline of the opening of the catheter shaft is located no more than 0.80 inches from the first end of the inflatable balloon.

10. The catheter of claim 1, wherein the inflatable balloon is a multi-layered inflatable balloon including a first balloon layer and a second sheath layer.

11. The catheter of claim 1, wherein the inflatable balloon includes ribs.

12. The catheter of claim 11, wherein the ribs extend in a direction generally parallel to the catheter shaft.

13. The catheter of claim 11, wherein the catheter shaft includes grooves sized to receive the ribs.

14. The catheter of claim 10, wherein the inflatable balloon includes ribs formed in the balloon layer, the ribs extending in a direction generally parallel to the catheter shaft, the ribs being sized to fit within grooves formed in the catheter shaft.

15. A kit, comprising:
a) a catheter including:
i) a catheter shaft defining a fluid lumen and a capillary lumen;
ii) a balloon in fluid communication with the capillary lumen;
iii) an end piece having first and second ports in fluid communication with the fluid and capillary lumens, respectively;
b) a syringe pre-filled with less than about 7 cc of fluid, the syringe being constructed to inject the fluid into the second port of the end piece to inflate the balloon.

16. The kit of claim 15, wherein the balloon has length defined between a first end and a second end, the length of the balloon being about 0.40 inches or less.

17. The kit of claim 15, wherein the inflatable balloon defines a balloon cavity, the balloon cavity being configured to receive less than about 7 cc of fluid.

18. The kit of claim 17, wherein the balloon cavity is configured to receive about 6 cc of fluid.

19. A method of manufacturing a catheter, the method comprising the steps of:
a) providing a catheter shaft; and
b) attaching an inflatable retention balloon to the catheter shaft at a first end of the balloon and at a second end of the balloon such that the balloon has a length between the first end and second end of about 0.40 inches or less.

20. The method of claim 19, wherein the step of attaching the inflatable retention balloon includes attaching a retention balloon having a diameter of about 0.80 inches to 1.1 inches when inflated.
